(19) United States
(12) Patent Application Publication
(10) Pub. No.: US 2023/0390314 A1
(43) Pub. Date: Dec. 7, 2023

(54) METABOLIC ACTIVATORS FOR ENHANCING SPERM CAPACITATION IN MAMMALS

(71) Applicant: UNIVERSIDADE DO PORTO, Porto (PT)

(72) Inventors: Marco Aurélio GOUVEIA ALVES, Porto (PT); Carlos Pedro FONTES OLIVEIRA, Porto (PT); David MARTÍN HIDALGO, Porto (PT)

(21) Appl. No.: 18/033,345

(22) PCT Filed: Oct. 20, 2021
(86) PCT No.: PCT/IB2021/059686

§ 371 Date: Apr. 23, 2023

(30) Foreign Application Priority Data
Oct. 22, 2020 (PT) 116847

(51) Int. Cl. A61K 31/69; C12N 5/076; C12N 5/075; C12N 5/073

(52) U.S. Cl. CPC A61K 31/69; C12N 5/061; C12N 5/0609; C12N 5/0604; C12N 2501/727

(57) ABSTRACT

The present invention relates to a SIRT1 activator for use in promoting and/or enhancing sperm capacitation process in mammals, a specific process for these cells. The disclosure also relates to a compound and a kit comprising the SIRT1 activator. The present invention further discloses the use of a commercially available SIRT1 activator, YK-3-237, as an additive or as a new ingredient to any sperm media already existing in the market to promote the sperm capacitation process, promoting fertilization in mammals.

### FIELD OF THE INVENTION

The present invention relates to the field of biomedicine, particularly to the field of compounds for use in assisted reproductive technologies (ART), namely in vitro fertilization and artificial insemination.

### BACKGROUND OF THE INVENTION

Human infertility has been recognized as a health problem spreading worldwide by the World Health Organization (WHO). As consequence, reproductive counsel in assisted reproduction clinics is blossoming. Indeed, infertility affects 1 in 6 couples seeking for a child (Sharlip et al., 2002) and the male factor contributes up to 50% of the cases of infertility (Agarwal et al., 2015). After ejaculation spermatozoa do not have fertility potential; they are required to go under a process named “capacitation” in order to be able to fertilize the oocyte. Historically, protein tyrosine phosphorylation has been used as “hallmark” of sperm capacitation status (Visconti et al., 1995a).

Men display a particular case of sperm capacitation behaviour. For instance, some men's ejaculates capacitate very early and others capacitate late in comparison with the average of the population (Ostermeier et al., 2018). Although there are timing differences on men ability to undergo through capacitation process (Hoshi et al., 1990), the timing (early, average or late capacitation status) is consistent within the individual (Ostermeier et al., 2018).

Human infertility is a health problem spread worldwide where 1 of each 6 couples seeking for a child face problems to achieve pregnancy. Thus, the interest on Assisted Reproductive Technologies (ART) is growing exponentially in the last years and is expected to continue to increase in the coming years. Moreover, current procedures to discriminate ejaculate fertility potential are not sufficient to stratify sperm capacity since ejaculate with normal seminogram parameters can be subfertile. Sperm capacitation is a pre-requisite in order to achieve fertility potential. Without capacitation there is no fertilization. Interestingly, there is variation among ejaculates of different men to achieve capacitation status but it is consistent within the individual.

In the international context of constant growing of couples looking for reproductive counsel and where 50% of infertility cases are associated to a male factor, there is a need for developing new media and methods to increase sperm functions to enhance the outcome of assisted reproductive technologies (ART). ART basically consists on collecting and handling oocytes, sperm and embryos in in vitro conditions designed with the aim to increase the reproductive outcome. Between the most used ART procedures are: i) in vitro fertilization (IVF), which consists in the co-incubation of sperm and oocyte in a dish (in vitro) to increase the chances of fertilization to occur; ii) intracytoplasmic sperm injection (ICSI), a technique for in vitro fertilization in which an individual sperm cell is introduced into an egg cell; iii) intrauterine insemination (IUI), by far the most simple ART procedure which involves placing sperm inside a woman's uterus close to the fallopian tubes in order to increase the chances of conceiving; iiii) cryopreservation, technology that allows storage of gametes and embryos at −180° C. for an unlimited period of time.

In fact, the current ART protocols are developed from the works provided in 1951 by two investigators who independently settled the cornerstone for the development of in vitro fertilization (IVF) (Austin, 1951; Chang, 1951). Interestingly, both authors observed that spermatozoa deposited into the oviduct were not able to fertilize the oocyte. Sperm has to reside for a period of time in the female tract in order to acquire fertility potential. Later on, these physiological changes that sperm must undergo through their transit along the female tract was named sperm capacitation (Austin, 1952).

Scientific community showed that calcium, bicarbonate and a cholesterol acceptor are needed to induce sperm capacitation in in vitro conditions. Spermatozoa as any other kind of cell regulate their functions by different intracellular pathways. Thus, it has been shown that a cholesterol acceptor in the sperm media is needed to increase sperm plasma membrane fluidity. By another hand calcium (Ca²⁺) and bicarbonate (HCO₃⁻) are needed to increase intracellular cAMP through activation of soluble adenyl cyclase (Adyc10). Subsequently, high levels of cAMP activate PKA protein, which triggers a cascade of events downstream that finalize with the phosphorylation of tyrosine residues that historically has been used as “hallmark” of sperm capacitation status (Visconti et al., 1995a; Visconti et al., 1995b; Mahony and Gwathmey, 1999; Tardif et al., 2001; Bravo et al., 2005).

Different species need different substrates to activate or sustain capacitation events. For instance, mouse and human spermatozoa capacitating media include glucose where this energy substrate is avoided on bovine capacitation media because it blocks sperm capacitation (Parrish et al., 1989; Williams and Ford, 2001; Travis et al., 2004). Differences have also been found between species in the kinetic to achieve maximum levels of phosphorylation of tyrosine residues: an hour in mouse, 6-18 hours in human or 4 hours in boar.

A single ejaculate contains different subpopulations of spermatozoa that achieve capacitation at different time points. This evolutive strategy allows spermatozoa to maximize the fertilizing timing ability at the site of fertilization (Fraser, 1999). Nevertheless, men are a particular case of sperm capacitation behaviour. Some ejaculated sperm capacitate very early while others capacitate late in comparison with the average of the population (Ostermeier et al., 2018). Although there are timing differences on men ability to undergo through capacitation process (Hoshi et al., 1990), timing (early, average or late capacitation status) is consistent within the individual (Ostermeier et al., 2018). This last work highlighted the issues associated to dysregulation of men spermatozoa capacitation process, in either early or late group (Ostermeier et al., 2018). Both groups present disadvantages when ART are applied to overcome fertility issues particularly because the timing associated to different ART procedures are standardized and fixed (Ostermeier et al., 2018). Standardized timing protocols do not contemplate the diversity of men ejaculates in terms of capacitation synchronization events. Protocols standardization precisely fails on those patients with fertility disorder seeking for ART to bypass their reproductive issues.

Moreover, it is a fact that current classic sperm analysis protocols are not able to predict the fertilizing capacity of an ejaculate since men with normal sperm parameters can be subfertile or even infertile. Therefore, new standardized protocols of ejaculates analysis should be established to predict the success of fertilization (Wang and Swerdloff, 2014). It has been proposed that the major problem is not associated with sperm quality determined by classical spermiogram but that men infertility might be due to spermatozoa inability to undergo the capacitation process. In line with this trend, Travis' laboratory has been working on the classification of human ejaculate ability to achieve capacitation trough monosialotetrahexosylganglioside (GM1) localization patterns on human spermatozoa (Cardona et al., 2017; Moody et al., 2017; Ostermeier et al., 2018). Plasma membrane sterol removal induced during the process of capacitation was reported to be associated to an enrichment of ganglioside GM1 (Cohen et al., 2014). Thus, depending on ejaculates' GM1 patterns a correlation was established with their probability to achieve pregnancy (Schinfeld et al., 2018).

In summary, different stimulus and signalling pathways are highly orchestrated with the aim of increasing spermatozoa chances to fertilize an oocyte at the proper timing. Any lapse on the timing of the sequential steps that allow spermatozoa to be able to fertilize will lead to fertility problems. Fortunately, the effectiveness or ART has enhanced along the last years allowing clinicians to reduce the number of embryos transferred by ART cycle, thus avoiding the negative impact of a multiple pregnancy (Kushnir et al., 2017). Notwithstanding the increase of success there is still an ample scope for an improvement. For example, in Europe the average in vitro fertilization (IVF) success is 28.5% where intracytoplasmic sperm injection (ICSI) is 26.2% and intrauterine insemination (IUI) is 7.8% (De Geyter et al., 2020).

There is therefore the need for developments that can control fertilization, a critical process for fertility, as is the case of the present invention.

Regardless cryopreservation, IVF, ICSI and IUI are among the most used the procedures by clinicians to counteract sub-fertility issues. Thus, at present, there is a trend where ICSI is the prevailing ART chosen by clinicians in detriment of IVF and IUI (De Geyter et al., 2020). Nevertheless, IUI is by far the easiest, more economic and women friendly method. After ejaculation, billions of sperm get into the female reproductive tract, approximately 1-10 thousand sperm are present in the isthmus, and only 10-100 sperm may be in the ampulla (where fertilization takes place) after 4-12 hours. However, these numbers might be reduced in men with subfertility or infertility as those that are under fertility treatment. Thus, one of most simplified protocol of ART is IUI that consist of increasing sperm number into the uterus to enhance the chances to encounter the oocyte. Regarding to ART associated cost, it is estimated that IUI is between 3 to 6 times cheaper than IVF/ICSI (Babigumira et al., 2018). But IUI also presents benefits beyond the economic point of view, particularly to the male counterpart. Thus, women under IVF/ICSI cycle suffer aggressive hormonal therapy and invasive and surgical procedures to pick up oocytes that later on will be used to create an embryo in in vitro conditions. Eventually, it will be transferred back to the women with all the distress and emotional cost associated to the whole process.

As mentioned before, nowadays first line of infertility treatment befall IVF/ICSI procedure. Nevertheless, the prevalence of IVF/ICSI over the most economic, simple and female friendly protocol as IUI, it is not always very well justified (Homburg, 2003). It seems that the election of IVF/ICSI as the primary technology to be used to treat infertility is due to a debatable higher success rate. The indiscriminate use of ICSI is only sustained because a mild improvement of the effectiveness of the procedure (less attempts are needed in order to fertilize an oocyte) at the expenses of others more simple and cheaper protocols as IUI for instance.

It has been shown that when couples under reproductive counsel decide to proceed with a heterologous insemination (meaning: perform an artificial insemination with the semen of a donor who is not the woman's partner), an improvement of 53% of fertility was reported. This last point emphasizes that an improvement of sperm quality increases the fertility chances when IUI is used as the selected ART to surpass subfertility. Any outcome improvement associated to any ART will be very helpful for these couples seeking for ART counsel but refrain to use sperm donors to the benefit of the use of their own genetic material. In other words, although there is an established system to provide spermatozoa from healthy donors, mostly from cryobanks, it is well known that couples under ART treatment only use it when there are no other possible chances to use their own genetic material. Hence, any chance to increase the opportunity to achieve pregnancy using their own genetic material will be very welcome.

Due to the worldwide reproductive health problems, different procedures have been developed with the aim to bypass infertility. Assisted reproduction new era started in the 40's when first artificial insemination (IUI) was described in humans. 30 years later, the first human in vitro fertilization (IVF) baby born on 1978. Later on, it a procedure named intracytoplasmic sperm injection (ICSI) was developed. With ICSI can be bypassed even the lack of sperm motility or sperm capacitation consisting in the introduction of the sperm into the oocyte. The first ICSI baby born in 1992. These 3 procedures are the most used nowadays in reproductive clinics, thus it can be categorized from less to more complex 1-IUI, 2-IVF and 3-ICSI. The choice of one or other procedure is based on the special characteristic of the couple under reproductive counsel. Nevertheless, currently clinicians are opting to choose ICSI as first tool to deal with men subfertility because it can be obtained the expected results, fertilization, with less attempts. However, ICSI by far is the most expensive (up to 6 times in comparison with IUI) and less women friendly between them due to the harsh hormonal treatment and medical intervention associated to it.

Sirtuins (SIRT1-7) are a family of NAD+-dependent deacetylases which catalyze post-translational modifications of proteins. YK-3-237 is commercially available (Tocris and Cayman) as an activator or sirtuin-1. Sirtuin are evolutionally conserved and belong to class Ill histone deacetylases (HDACs), comprising seven members. The sirtuin-mediated deacetylation reaction couples lysine deacetylation to NAD+ (nicotinamide adenine dinucleotide) hydrolysis releasing NAM (nicotinamide), Acetyl-ADP (adenosine diphosphate) ribose and has a consequence of deacetylation of the target protein (Rato et al., 2016).

Sirtuins have been associated with aging and longevity (Vachharajani et al., 2016), linked to processes like apoptosis and cell survival (Alcendor et al., 2004), fatty acid oxidation (Purushotham et al., 2009), DNA repair, development and neuroprotection (Donmez and Outeiro, 2013) and mitochondrial biogenesis (Brenmoehl and Hoeflich, 2013).

Sirtuins play a role on gametes and embryo and functions (Rato et al., 2016; Tatone et al., 2018; Meroni et al., 2019).

The activator YK-3-237 is a specific activator for SIRT1. So far it has been used to inhibit the proliferation of breast cancer cell (Yi et al., 2013), nevertheless the activator promotes renal fibroblast and aggravates renal fibrogenesis (Ponnusamy et al., 2015).

The safety or teratogenicity effects on embryos produced by the use of sperm capacitated using YK-3-237 has not yet been ruled out and cannot be predicted.

Nevertheless, some publications described the use of YK-3-237 in other types of cells (Yi et al., 2013; Ponnusamy et al., 2015). YK-3-237 has an antiproliferative effect on breast cancer cells (Yi et al., 2013). By another hand, negative effects have been associated to YK-3-237 because promotes renal fibroblast activation and aggravates renal fibrogenesis (Ponnusamy et al., 2015).

A recent publication (Patricia Braga, 2019) discloses a study of activator YK-3-237 in Sertoli cells, disclosing its role in spermatogenesis of mice. Surprisingly, the present invention discloses for the first time the role of SIRT1 activators in mammals capacitation which is a specific process of spermatozoa.

Effects of Sirt-1 activation is specific for the processes in study. For example, Sirt-1 activation by resveratrol is associated with protection against oxidative stress in ovaries (Ochiai and Kuroda, 2020) and suggested as beneficial for in vitro embryo production (Adamkova et al., 2017).

Any ART procedure that involves male gamete handling is a candidate to use this activator to increase the percentage of spermatozoa that achieve capacitation status, a process specific to these cells and necessary for fertilization, therefore increasing the chances that fertilization occurs.

Regarding veterinary applications, for example, there are some unsuccessful applications in vitro fertilization (IVF) on horses despite decades of intense research (Hinrichs, 2013). Equine IVF fails because stallion spermatozoa do not capacitate properly on in-vitro media.

Most of the technologies that try to improve sperm fertility potential have been focused on the improvement of sperm motility and/or hypermotility. For example, several methods were patented where there is an increase of hypermotility associated to an improvement of fertilization rate (WO 2017/173391 AI, U.S. Pat. No. 10,470,798 B1) or by the use of a metabolic enhancer (mTOR activator) achieved to improve the sperm velocity (WO2019025961).

Little has been done regarding sperm capacitation status, although the importance of this process on the fertilization process has been highlighted by Travis's Lab to rate the time that men ejaculates need to acquire capacitation status and associating the timing-rate to the fertility potential (U.S. Pat. No. 7,160,676). Moreover, Bronson and Huntington described a method of screening for infertility of sperm based on fibronectin antibody binding levels (U.S. Pat. No. 5,256,539). Nevertheless, other disclosure (PCT/US03/16669) used CRISP polypeptide to inhibit sperm capacitation, tyrosine residues phosphorylation, acrosome reaction and fertilization process. In the same line, (PTC/BF20004/004912) was proposed the use of specific proteins (such as fibronectin and angiotensin II) to respectively conserve sperm in a non-capacitated or non-activated stated.

U.S. Pat. No. 5,834,225 discloses a method to increase sperm capacitation (examined with chlortetracycline fluorescence assay) using hydrogen peroxide or other reactive oxygen sources.

These facts are outlined to enhance the problem solved by the present invention.

### General Description of the Invention

The present invention discloses the use of YK-3-237 in sperm with the aim to enhance spermatozoa capacitation, a cell-specific process that happens in vivo along their travel through the female reproductive track seeking for an oocyte. The use of YK-3-237 as additive to human sperm media increase the levels of tyrosine phosphorylation that has been historically used as hallmark of the sperm capacitation process and might indicate higher chances that fertilization occurs.

The present disclosure describes the use of commercially available YK-3-237 to induce and synchronize sperm capacitation status defined as displaying high levels of tyrosine residues phosphorylation.

The present invention intends to solve the inability to achieve capacitation as well as those cases were the capacitation status is delayed.

In an embodiment, subfertile men with problems associated to their inability to achieve sperm capacitation status will have a second chance to achieve fertility through a regular ART but adding an extra step; for example: co-incubation of spermatozoa with the drug herein disclosed before IUI is performed or along the incubation period during the capacitation period previous to an IVF procedure.

In a further embodiment, delayed capacitated spermatozoa will meet the oocyte in sub-optimal conditions (ageing oocytes) with lower chances to go further in the fertilization and development process. Thus, co-incubation of spermatozoa with YK-3-237 or B-[2-methoxy-5-[(1E)-3-oxo-3-(3,4,5-trimethoxyphenyl)-1-propen-1-yl]phenyl]-boronic acid which hasten the intracellular signalling that spermatozoa needs in order to be able to fertilize, mitigating the oocyte waiting time, thus increasing both gametes interaction in their best timing-conditions.

In brief, irrespective of sperm ability to achieve capacitated status (slow, average or delayed subject), here we describe an activator which synchronizes and enhances sperm ability to fertilize an oocyte, and its application to a broad assisted reproductive procedures currently used on reproductive clinics worldwide.

In a particular embodiment, this approach is applied when fertile men are used as semen donors. For instance, it is applied when ART cycles use low quality oocytes with lower fertilization time windows.

In an embodiment, this invention discloses a product which enhances sperm capacitation process. It is indicated to couples with problems to conceive a child associated to male factor; specially in those cases of male idiopathic infertility and more specifically in those reported cases of male infertility associated to sperm capacitation disorders.

In a further embodiment, the present invention discloses the use of YK-3-237 to synchronize capacitation events at earlier time and increase up to 3 folds the tyrosine phosphorylation levels (Please see FIGS. 1A, 2A and 3A).

In a further embodiment, the present invention discloses the use of the commercially available activator YK-3-237 as an additive or as a new ingredient to any sperm media already existing in the market to promote the sperm capacitation process increasing the chances that fertilization occurs.

In an embodiment, the present invention is directed to mammals. The results herein provided include a wide spectrum of species that present fertility problems associated to the inability to display sperm capacitation status, with special interest on species with economic interest and endangered/exotic animals. Thus, for instance, animal breeding selection is focused on producing offspring with a desirable phenotype. However, this selection pressure is not focused on reproductive parameters which might be affected implying that several ART attempts, for example artificial inseminations, must be performed to achieve fertility with the rise of the cost associated with the procedure. For instance, animal farm industry is suitable to exploit the beneficial effects of enhanced sperm capacitation process. Equine IVF fails mainly due to incomplete activation of spermatozoa due to inadequate capacitating media (Leemans et al., 2016).

In one embodiment, the present invention discloses the use of commercially available YK-3-237 to a wide spectrum of sperm media used to performed artificial insemination, intra uterine insemination, IVF, etc, in summary to any ART protocol that imply the collection, storage and/or processing sperm in any specie.

The present disclosure contributes to the development of the welfare of society by:
i) increasing the chances to conceive a child in couples where the men have problems associated with sperm capacitation process;
ii) promoting the use of IUI procedure in combination with YK-3-237 which is a cheaper and user friendlier ART procedure than others; which in addition increasing the affordability of ART counsel in the most economically depressed social sectors.

The present disclosure differs from current products since they are mostly focused on the improvement of sperm motility forgotten the pivotal role of sperm capacitation on the fertilization process. It should be mention that the spermatozoa displaying the best motile parameters will not be able to fertilize an oocyte if capacitation has not achieved. Hence, the activator YK-3-237 overcome subfertility associated to sperm capacitation disorder. Consequently, YK-3-237 provides an alternative to those couples struggling with infertility problem associated to sperm capacitation issue where they now might opt for more physiological and cheaper method (IUI and IVF) before to consider Intracytoplasmic Sperm Injection (ICSI).

In a first embodiment, the present invention discloses a SIRT1 activator for use in promoting and/or enhancing sperm capacitation process in mammals.

In a second embodiment the SIRT1 activator is at a concentration from 5-30 μM.

In a further embodiment SIRT1 activator is at a concentration of 10 μM.

In a further embodiment the SIRT1 activator is B-[2-Methoxy-5-[(1E)-3-oxo-3-(3,4,5-trimethoxyphenyl)-1-propen-1-yl]phenyl]boronic acid or YK-3-237.

In a further embodiment SIRT1 activator is used in assisted reproductive technology.

In a further embodiment SIRT1 activator is used in assisted reproductive technology carried out with low quality oocytes.

In a further embodiment SIRT1 activator is used in assisted reproductive technology, namely intra-uterine insemination, in vitro fertilization or intracytoplasmic sperm injection.

In a further embodiment SIRT1 activator is used in gradient sperm selection.

In a further embodiment SIRT1 activator is used in idiopathic infertility or in male infertility associated to sperm capacitation disorders.

In a further embodiment SIRT1 activator is combined with a further sperm promoter and/or enhancer.

In a further embodiment, the SIRT1 activator is combined with a commercially available sperm media, preferably sperm washing media, cryopreservation media, thawing media or combinations thereof.

In a further embodiment, the SIRT1 activator is combined with a media comprising spermatozoa previously washed and diluted.

In a further embodiment, the SIRT1 activator induces maximum tyrosine phosphorylation levels on human spermatozoa after incubation in a capacitating media containing HCO₃ and a sterol removal component.

In a further embodiment, the SIRT1 activator is for use in humans.

In a further embodiment, the SIRT1 activator is for use in non-human mammals, particularly equines, more particularly in horses.

In a further embodiment, the disclosure encompasses a pharmaceutical compound comprising the SIRT1 activator, in particular a YK-3-237 activator.

In a last embodiment, the disclosure comprising a kit comprising a SIRT1 activator, in particular a YK-3-237 activator.

### DETAILED DESCRIPTION OF FIGURES

FIG. 1. Effect of YK-3-237 on protein phospho-tyrosine level in human sperm. Human spermatozoa were incubated for 6 hours at 37° C. in capacitating conditions (BWW-modified media supplemented with HCO₃ 25 mM and BSA 26 mg/mL) in presence or absence of different concentrations of YK-3-237. A) Left panel: a representative western blot using anti-phospho-tyrosine substrate of human sperm incubated at different concentrations of YK-3-237 in capacitating conditions. Right panel: western blots were analyzed using ImageJ (n=7). For comparison between blots, pixels for each lane were quantified and normalized using the CAP (0 μM) lane as reference (100%). B) Sperm viability of human spermatozoa incubated at different concentrations of YK-3-237 determined by eosine-nigrosine stain (n=4). Dotted line shows initial sperm viability before to initiate sperm incubation. C) Example of human spermatozoa stained with eosine-nigrosine stain. Sperm with white background it is an example of alive spermatozoa where spermatozoa with pink background shows a dead spermatozoa. Bars represent the average±SEM. Data were analyzed statistically by one-way analysis of variance (ANOVA). Differences between concentrations were analyzed by Tukey post-hoc test, *p<0.05 and ***p<0.0005 indicate differences versus capacitating (0 μM) conditions.

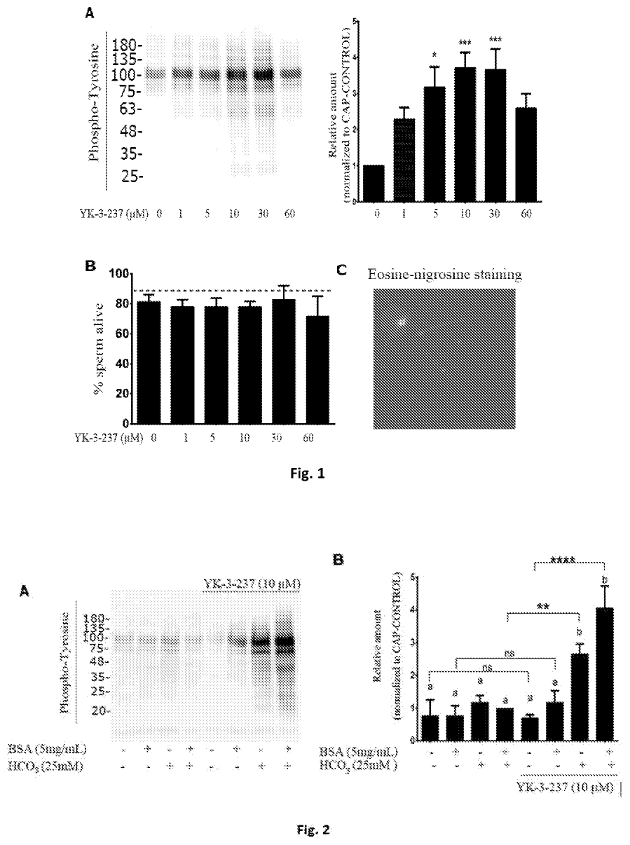

FIG. 2. Role of HCO₃ and BSA on the induction of tyrosine phosphorylation level by YK-3-237 (10 μM) in human sperm. Human spermatozoa were incubated for 6 hours at 37° C. in different conditions that support or not human sperm capacitation. A) A representative western blot using anti-phospho-tyrosine substrate of human sperm incubated in different conditions (presence or absence of HCO₃⁻ 25 mM and BSA 26 mg/mL) with or without YK-3-237 for 6 hours at 37° C. (n=3). B) Western blots were analyzed using ImageJ (n=3). For comparison between blots, pixels for each lane were quantified and normalized using the CAP (YK-3-237 0 μM) lane as reference (100%). Bars represent the average±SEM. Data were analyzed statistically by one-way analysis of variance (ANOVA). Tukey post-hoc test was used to analyzed differences between the presence of absence of YK-3-237 (10 μM), **p<0.005 and ****p<0.0001 indicate differences versus YK-3-237 (10 μM) conditions. Different superscript a,b show differences between treatments p<0.05.

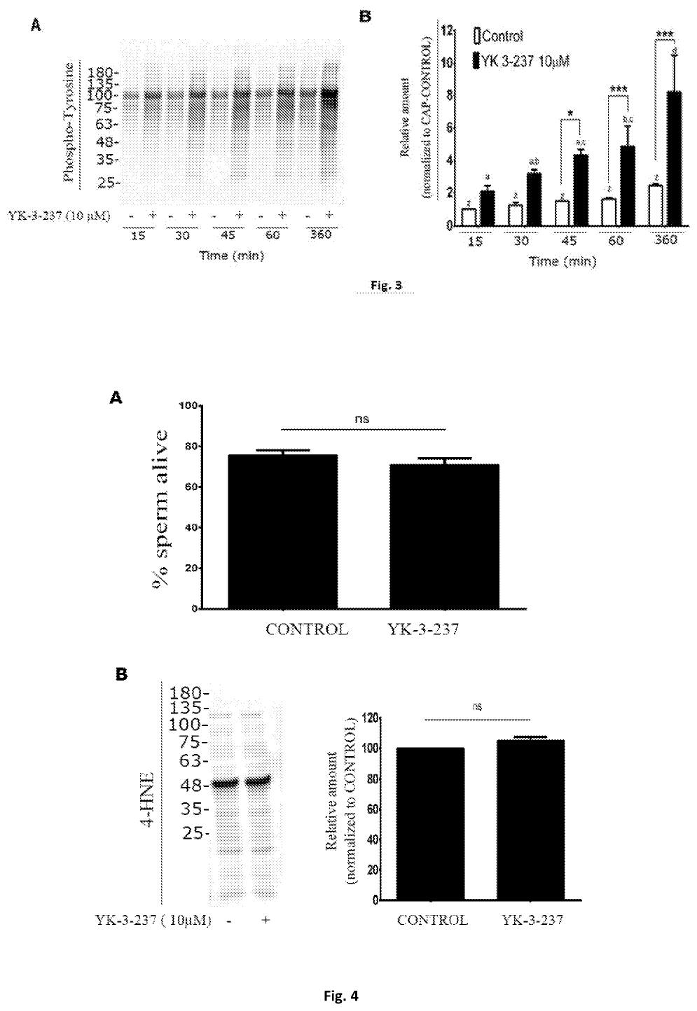

FIG. 3. Time curve of tyrosine phosphorylation levels of human spermatozoa incubated in presence or absence of YK-3-237 (10 μM). A) A representative western blot using anti-phospho-tyrosine substrate of human sperm incubated at different time points in capacitating conditions (HCO₃−25 mM and BSA 26 mg/mL) in presence or absence of YK-3-237 (10 μM) (n=3). For comparison between blots, pixels for each lane were quantified and normalized using the CAP (YK-3-237 0 μM) lane as reference (100%). Bars represent the average±SEM. Data were analyzed statistically by one-way analysis of variance (ANOVA). Tukey post-hoc test was used to analyzed differences between the presence of absence of YK-3-237 (10 μM), **p<0.005 and ***p<0.0005 indicate differences versus YK-3-237 (10 μM) conditions at different time points. Different superscript a,b,c show differences between treatments YK-3-237 (10 μM) along the time. Different superscript z,y show differences between control conditions (0 μM) along the time.

FIG. 4. Effect of YK-3-237 (10 μM) on human sperm viability and lipid peroxidation levels (4-HNE). Human spermatozoa were incubated for 6 hours at 37° C. in conditions that support sperm capacitation (HCO₃25 mM and BSA 26 mg/mL) in presence or absence of YK-3-237 (10 μM). A) Sperm viability was determined by eosine-nigrosine of spermatozoa incubated in presence or absence of YK-3-237 (10 μM) for 6 hours at 37° C. (n=11). B) Left panel: a representative western blot using anti-4 Hydroxynonenal (4-HNE) antibody used to measure lipid peroxidation levels (n=4). B) Western blots were analyzed using ImageJ (n=4). Right panel: for comparison between blots, pixels for each lane were quantified and normalized using the CAP (YK-3-237 0 μM) lane as reference. Bars represent the average±SEM. Differences between conditions were analyzed using two-tailed, paired t-test *p<0.05 shows differences versus YK-3-237 (10 μM).

### DETAILED DESCRIPTION OF THE INVENTION

The present invention enhances the sperm capacitation process through the use of a metabolic activator. The SIRT1 activator has the ability to synchronize sperm capacitation process independently of the initial ejaculate status.

Moreover, the SIRT1 activator brings forward sperm capacitation events (tyrosine phosphorylation). After 6 hours of capacitation in combination with the metabolic activator, tyrosine phosphorylation levels triplicate in comparison with control, showing that more spermatozoa might be ready to fertilize the oocyte.

In an embodiment, the present disclosure uses a SIRT1 activator to enhance sperm capacitation for further use in reproductive technologies (ART). ART consists in collecting and handling oocytes, sperm and embryos in in vitro conditions designed with the aim of increasing the reproductive outcome.

In another embodiment, the activator is used in combination with standardized IUI protocol to increase the number of IUI performed as expenses of IVF/ICSI. These last 2 procedures are more expensive, complex and less friendly to women.

In a particular embodiment, a specific SIRT1 activator, YK-3-237 is used in others ART as IVF with the aim to maximize the number of spermatozoa capacitated increasing thus the chances that fertilization occurs.

The present invention is also for veterinary application. The present results show that it is to be applied to a wide spectrum of species with fertility problems associated to inability to display sperm capacitation status, with special interest on those species with economic interest and endangered/exotic animals. For instance, animal farm industry is suitable to exploit the beneficial effects of enhance sperm capacitation process; for example, equine in vitro fertilization fails mainly due to incomplete activation of spermatozoa because of inadequate capacitating media (Leemans et al., 2016).

In an embodiment, the activator is used in any ART which implies obtention and processing sperm in in vitro conditions.

The present invention provides the use of an activator to enhance the sperm capacitation process. The activator synchronizes sperm capacitation process independently of the initial ejaculate status. Thus, with the present activator, which is also a metabolic enhancer, it is possible to promote capacitation events and increase the number of spermatozoa ready to fertilize an oocyte. This invention is useful to be applied in spermatozoa from men unable to conceive a child due to problems associated to sperm capacitation events.

In an embodiment, the activator is used in combination with standardized intrauterine insemination (IUI) protocol to increase the number of IUI performed as expenses of IVF/ICSI. These last 2 procedures are more expensive, complex protocol and less friendly to women.

The present invention is also for veterinary use. For instance, animal farm industry is suitable to exploit the beneficial effects of enhancing sperm capacitation process. The equine sector does not have available a successful in vitro fertilization protocol due to an incomplete activation of spermatozoa associated to an inadequate capacitating media. Therefore, the use of the activator disclosed in the present disclosure provides beneficial effects for this specific industry.

Here, we provide an invention based on the supplementation of a metabolic enhancer to induce maximum tyrosine phosphorylation levels on human spermatozoa after 6 hours of co-incubation in a defined capacitating media containing HCO₃ and a sterol removal. Both components are universal between all the commercial in vitro fertilization media available nowadays. Thus, after 6 hours of incubation in capacitating conditions (bicarbonate and BSA), the best concentration of YK-3-237 which enhanced tyrosine phosphorylation levels was of 10 μM (Figure-la). Moreover, none of the concentration tested had detrimental effect of sperm viability (Figure-1B). After that, the inventors decided to test with one of the components of capacitating media (bicarbonate or BSA) to determine which one is responsible for the increase of tyrosine phosphorylation in presence of YK-3-237 (FIG. 2). Although co-incubation of YK-3-237 in presence of bicarbonate increases the tyrosine phosphorylation levels, this increase was further improved when there was a co-incubation of YK-3-237 in a complete capacitating media containing bicarbonate and BSA (Figure-2). Interestingly, after just 1 hour of co-incubation of spermatozoa with YK-3-237 in capacitating conditions, we achieved the same levels of tyrosine phosphorylation as control samples after 6 hours (Figure-3) illustrating that we found a faster way to induce and achieve capacitation status. Moreover, after 6 hours of incubation, YK-3-237 tripled the levels of tyrosine phosphorylation in comparison with control 6 hours (Figure-3) which shows that more spermatozoa might be ready for fertilization. This means that the present invention identified a way to have more spermatozoa ready for fertilization in a faster way. The inventors checked the spermatozoa viability for the best/selected concentration of YK-3-237, 10 μM, and found that it did not affect sperm viability nor induced any change on markers associated to oxidative stress (lipid peroxidation (4-HNE)) even after 6 hours (Figure-4).

YK-3-237 allows to hasten the capacitation signaling pathway understood as increased levels of protein tyrosine phosphorylation. Currently, the gold standard technique to measure sperm capacitation status or ability to undergo capacitation process is determining the protein tyrosine phosphorylation levels by western blotting. Although western blotting is a standardized procedure used in molecular biology there is not a routine probe performed in fertility clinics. Moreover, it is not characterized by obtaining results very fast, an average of 3 days entailing: incubation in capacitation conditions by at least 6 hours, protein extraction and concentration determination, plus the timing involved in the western blotting procedure, are needed. Using the activator YK-3-237, there is no need to evaluate the ability of the ejaculate to capacitate.

Hence, the present invention brings forward this waiting time and the present findings show it is possible to avoid the uncomfortable proceeding of a second semen donation and the annoying displacement to the fertility clinics.

In a particular embodiment, the present invention discloses not only an increase of the protein tyrosine phosphorylation levels but so bring forward this event. Within one hour of co-incubation we achieved the same levels that are only reached after 6 hours of incubation in control capacitating conditions. Moreover, after 6 hours of sperm co-incubation with YK-3-237, the tyrosine levels are tripled in comparison with control. Consequently, the present disclosure increased the number of spermatozoa in an optimal status to accomplish fertilization increasing their chances to fertilize and oocyte. One cannot neglect the use of YK-3-237 (which enhances capacitation events associated) in combination with other procedures that improve sperm motility as for example with a method using mTOR activators (MHY-1485).

In one embodiment YK-3-237 is added to the media where spermatozoa are washed and diluted before to perform an intrauterine insemination.

In a further embodiment, YK-3-237 is added to the sperm capacitation media previously to the coincubation with the oocyte. Although there is an established system to provide spermatozoa from healthy donors, mostly from cryobanks, it is well known that couples under ART treatment only use it when there are no other possible chances to use their own genetic material. Hence, any chance to increase the opportunity to achieve pregnancy using their own genetic material, as the inventors disclose, will be very welcome.

In another embodiment, YK-3-237 is used alone or in combination with other sperm enhancers. For instance, YK-3-237 is used in combination with other methods which improve sperm motility that would further increase the sperm ability to fertilize and oocyte.

For sperm incubations Biggers-Whitten-Whittingham (BWW) media described by Biggers J D et al. (1971) with slightly modifications was used. BBW-washed (BWW-W): 94.5 mM NaCl, 4.8 mM KCl, 1.7 mM CaCl₂×2H2O, 1.17 mM KH₂PO₄, 1.22 mM MgSO₄×7H₂O, 20 mM HEPES. Non capacitation media (NC) contains all the component of BWW-washed plus 5.56 mM glucose and gentamycin 10 mg/mL. Capacitating media (CAP): contains all the component of NC plus 25 mM of HCO₃ and 26 mg of Bovine Serum Albumin (BSA). All the media were balanced to a final pH 7.2-7.4

For sperm preparation, semen samples were collected by masturbation into sterile cups after 2-4 days of sexual abstinence. Samples were left to liquefy for up 2 hours at 37° C. to complete liquefaction prior to processing as described below. Only ejaculates whose semen parameters (total fluid volume, sperm concentration, motility and morphology) meet the (2010) normality criteria established by the WHO (2010) were processed. Ejaculates were submitted to discontinuous gradient centrifugation (Sperm Wash Gradient Set (45% and 90%)) for 20 min at 300 g at room temperature. The bottom gradient layer (purified populations of highly motile sperm) was recovered and washed (500 g for 5 min at room temperature) with modified BWW-W media that does not contain any energy substrate. The pellet was then resuspended in 1 mL of non-capacitating (NC) media and sperm concentration was determined using a Neubauer counting chamber under an optical microscope (×100 magnification). Due to the low number of spermatozoa obtained in each ejaculate, different ejaculates were pooled for different assessments. Finally, depending on experimental design spermatozoa were diluted in 1 ml of capacitating (CAP media containing 26 mg/mL of BSA and 25 mM of bicarbonate) or NC at a final concentration of 20×106/ml for 6 hours at 37° C. in presence or absence of the activator. YK-3-237 was added at the referred concentration at time 0 of incubation and keep along the time of sperm incubation.

For determination of sperm viability, the instructions by the WHO (2010) were followed, sperm viability was assessed by eosin-nigrosin staining technique. Briefly, an aliquot of semen was mixed with an equal volume of eosin-nigrosin suspension. This suspension was used to make a smear on a glass slide. A total of 200 spermatozoa were counted in random fields under a bright-field microscope. Dead spermatozoa stained pink, as the loss of membrane integrity allows the cells to take up eosin, whereas live cells appear white. Nigrosin stains the background in a dark violet colour for a better visualization of the cells.

For the Western blot analysis, spermatozoa were washed twice in phosphate-buffered saline (PBS) by centrifugation at 5000 g for 3 min at room temperature. The pellet was resuspended in 2×Laemmli sample buffer and incubated over night at 4° C. The samples were then centrifuged again (10000 g, 15 min, 4° C.) and the protein concentration was measured using a Bio-Rad DC Protein Assay. Samples were boiled for 5 min at 95° C. in presence of 2-mercaptoethanol (2.5%), and 25 μg of each sample was subjected to 10% sodium dodecyl sulphate-polyacrylamide gel electrophoresis at constant voltage (100 V) before being transferred to PVDF membranes (Biorad semi-dry system, protocol mix for 8 min). The phosphorylation state of the sperm proteins was analysed by overnight incubation at 4° C. with an anti-phosphotyrosine monoclonal antibody (mAb; Clone 4G10, Millipore; diluted 1:3000, v/v, in Tris-buffered saline-Tween 20 solution (TBST) containing 3% milk). The levels of sperm lipid peroxidation was evaluated by overnight incubation at 4° C. with an anti-4-hydroxynonenal (4HNE) antibody (pAb; Millipore; diluted 1:3000, v/v, in Tris-buffered saline-Tween 20 solution (TBST) containing 3% BSA. Membranes incubated with anti-phosphotyrosine were incubated for 60 min at room temperature with a horseradish peroxidase (HRP)-conjugated secondary anti-mouse antibody (diluted 1:5000, v/v, in TBST containing or 3% milk). Membranes incubated with anti-4HNE were incubated for 60 min at room temperature with a horseradish peroxidase (HRP)-conjugated secondary anti-goat antibody (diluted 1:5000, v/v, in TBST containing or 3% BSA). Finally, membranes were washed 3 times for 5 min in TBST and incubated for 5 min with the Clarity™ Western ECL Substrate.

Membranes' fluorescence was read with the BioRad FX-Pro-plus (Bio-Rad, Hemel Hempstead, UK). Image analysis was conducted using the Image Lab vs 5.2 (Bio-Rad). Western blotting regions of interest (ROIs) used for quantification are indicated by a vertical bar on the right of the respective western blot. Images shown are representative of experiments repeated three times (n=3) using a pool of three different donors.

As will be clear to one skilled in the art, the present invention should not be limited to the embodiments described herein, and a number of changes are possible which remain within the scope of the present invention.

Of course, the preferred embodiments shown above are combinable, in the different possible forms, being herein avoided the repetition all such combinations.

### REFERENCES

Adamkova, K., Yi, Y. J., Petr, J., Zalmanova, T., Hoskova, K., Jelinkova, P., Moravec, J., Kralickova, M., Sutovsky, M., Sutovsky, P., Nevoral, J., 2017. SIRT1-dependent modulation of methylation and acetylation of histone H3 on lysine 9 (H3K9) in the zygotic pronuclei improves porcine embryo development. Journal of animal science and biotechnology, 8, 83. https://10.1186/s40104-017-0214-0.
Agarwal, A., Mulgund, A., Hamada, A., Chyatte, M. R., 2015. A unique view on male infertility around the globe. Reprod. Biol. Endocrinol., 13, 37. https://10.1186/s12958-015-0032-1.
Austin, C. R., 1951. Observations on the penetration of the sperm in the mammalian egg. Aust. J. Sci. Res. B, 4, 581-596. https://.
Austin, C. R., 1952. The capacitation of the mammalian sperm. Nature, 170, 326. https://10.1038/170326a0.
Babigumira, J. B., Sharara, F. I., Garrison, L. P., Jr., 2018. Projecting the potential impact of the Cap-Score on clinical pregnancy, live births, and medical costs in couples with unexplained infertility. J. Assist. Reprod. Genet., 35, 99-106. https://10.1007/s10815-017-1021-4.
Bravo, M. M., Aparicio, I. M., Garcia-Herreros, M., Gil, M. C., Pena, F. J., Garcia-Marin, L. J., 2005. Changes in tyrosine phosphorylation associated with true capacitation and capacitation-like state in boar spermatozoa. Mol. Reprod Dev, 71, 88-96. https://10.1002/mrd.20286 [doi].
Cardona, C., Neri, Q. V., Simpson, A. J., Moody, M. A., Ostermeier, G. C., Seaman, E. K., Paniza, T., Rosenwaks, Z., Palermo, G. D., Travis, A. J., 2017. Localization patterns of the ganglioside GM1 in human sperm are indicative of male fertility and independent of traditional semen measures. Mol. Reprod. Dev., 84, 423-435. https://10.1002/mrd.22803.
Chambers, G. M., Hoang, V. P., Illingworth, P. J., 2013. Socioeconomic disparities in access to ART treatment and the differential impact of a policy that increased consumer costs. Hum. Reprod., 28, 3111-3117. https://10.1093/humrep/det302.
Chambers, G. M., Sullivan, E. A., Ishihara, O., Chapman, M. G., Adamson, G. D., 2009. The economic impact of assisted reproductive technology: a review of selected developed countries. Fertil. Steril., 91, 2281-2294. https://10.1016/j.fertnstert.2009.04.029.
Chang, M. C., 1951. Fertilizing capacity of spermatozoa deposited into the fallopian tubes. Nature, 168, 697-698. https://.
Cohen, R., Buttke, D. E., Asano, A., Mukai, C., Nelson, J. L., Ren, D., Miller, R. J., Cohen-Kutner, M., Atlas, D., Travis, A. J., 2014. Lipid modulation of calcium flux through CaV2.3 regulates acrosome exocytosis and fertilization. Dev. Cell, 28, 310-321. https://10.1016/j.devcel.2014.01.005.
Collins, J., 2002. An international survey of the health economics of IVF and ICSI. Hum. Reprod. Update, 8, 265-277. https://10.1093/humupd/8.3.265.
De Geyter, C., Calhaz-Jorge, C., Kupka, M. S., Wyns, C., Mocanu, E., Motrenko, T., Scaravelli, G., Smeenk, J., Vidakovic, S., Goossens, V., 2020. ART in Europe, 2015: results generated from European registries by ESHRE. Human reproduction open, 2020, hoz038. https://10.1093/hropen/hoz038;
Hinrichs, K., 2012. Assisted reproduction techniques in the horse
547. Reprod Fertil. Dev, 25, 80-93. https://RD12263 [pii]; 10.1071/RD12263 [doi].
Homburg, R., 2003. The case for initial treatment with intrauterine insemination as opposed to in vitro fertilization for idiopathic infertility. Human fertility (Cambridge, England), 6, 122-124. https://10.1080/1464770312331369373.
Kushnir, V. A., Barad, D. H., Albertini, D. F., Darmon, S. K., Gleicher, N., 2017. Systematic review of worldwide trends in assisted reproductive technology 2004-2013. Reprod. Biol. Endocrinol., 15, 6. https://10.1186/s12958-016-0225-2.
Leemans, B., Gadella, B. M., Stout, T. A., De Schauwer, C., Nelis, H., Hoogewijs, M., Van Soom, A., 2016. Why doesn't conventional IVF work in the horse? The equine oviduct as a microenvironment for capacitation/fertilization. Reproduction, 152, R233-r245. https://10.1530/rep-16-0420.
Mahony, M. C., Gwathmey, T., 1999. Protein tyrosine phosphorylation during hyperactivated motility of cynomolgus monkey (Macaca fascicularis) spermatozoa. Biol. Reprod, 60, 1239-1243. https://.
Meroni, S. B., Galardo, M. N., Rindone, G., Gorga, A., Riera, M. F., Cigorraga, S. B., 2019. Molecular Mechanisms and Signaling Pathways Involved in Sertoli Cell Proliferation. Front. Endocrinol. (Lausanne), 1010.3389/fendo.2019.00224.
Moody, M. A., Cardona, C., Simpson, A. J., Smith, T. T., Travis, A. J., Ostermeier, G. C., 2017. Validation of a laboratory-developed test of human sperm capacitation. Mol. Reprod. Dev., 84, 408-422. https://10.1002/mrd.22801.
Ochiai, A., Kuroda, K., 2020. Preconception resveratrol intake against infertility: Friend or foe? Reprod. Med. Biol., 19, 107-113. https://10.1002/rmb2.12303.
Ostermeier, G. C., Cardona, C., Moody, M. A., Simpson, A. J., Mendoza, R., Seaman, E., Travis, A. J., 2018. Timing of sperm capacitation varies reproducibly among men. Mol. Reprod. Dev., 85, 387-396. https://10.1002/mrd.22972.
Ponnusamy, M., Zhuang, M. A., Zhou, X., Tolbert, E., Bayliss, G., Zhao, T. C., Zhuang, S., 2015. Activation of Sirtuin-1 Promotes Renal Fibroblast Activation and Aggravates Renal Fibrogenesis. J. Pharmacol. Exp. Ther., 354, 142-151. https://10.1124/jpet.115.224386.
Rato, L., Alves, M. G., Silva, B. M., Sousa, M., Oliveira, P. F., 2016. Sirtuins: Novel Players in Male Reproductive Health. Curr. Med. Chem., 23, 1084-1099. https://.
Schinfeld, J., Sharara, F., Morris, R., Palermo, G. D., Rosenwaks, Z., Seaman, E., Hirshberg, S., Cook, J., Cardona, C., Ostermeier, G. C., Travis, A. J., 2018. Cap-Score prospectively predicts probability of pregnancy. Mol. Reprod. Dev., 85, 654-664. https://10.1002/mrd.23057.
Sharlip, I. D., Jarow, J. P., Belker, A. M., Lipshultz, L. I., Sigman, M., Thomas, A. J., Schlegel, P. N., Howards, S. S., Nehra, A., Damewood, M. D., Overstreet, J. W., Sadovsky, R., 2002. Best practice policies for male infertility. Fertil. Steril., 77, 873-882. https://.
Tardif, S., Dube, C., Chevalier, S., Bailey, J. L., 2001. Capacitation is associated with tyrosine phosphorylation and tyrosine kinase-like activity of pig sperm proteins. Biol. Reprod., 65, 784-792. https://.
Tatone, C., Di Emidio, G., Barbonetti, A., Carta, G., Luciano, A. M., Falone, S., Amicarelli, F., 2018. Sirtuins in gamete biology and reproductive physiology: emerging roles and therapeutic potential in female and male infertility. Hum. Reprod. Update, 24, 267-289. https://10.1093/humupd/dmy003.
Visconti, P. E., Bailey, J. L., Moore, G. D., Pan, D., Olds-Clarke, P., Kopf, G. S., 1995a. Capacitation of mouse spermatozoa. I. Correlation between the capacitation state and protein tyrosine phosphorylation. Development, 121, 1129-1137. https://.
Visconti, P. E., Moore, G. D., Bailey, J. L., Leclerc, P., Connors, S. A., Pan, D., Olds-Clarke, P., Kopf, G. S., 1995b. Capacitation of mouse spermatozoa. II. Protein tyrosine phosphorylation and capacitation are regulated by a cAMP-dependent pathway. Development, 121, 1139-1150. https://.
Wang, C., Swerdloff, R. S., 2014. Limitations of semen analysis as a test of male fertility and anticipated needs from newer tests. Fertil. Steril., 102, 1502-1507. https://10.1016/j.fertnstert.2014.10.021.
Yi, Y. W., Kang, H. J., Kim, H. J., Kong, Y., Brown, M. L., Bae, I., 2013. Targeting mutant p53 by a SIRT1 activator YK-3-237 inhibits the proliferation of triple-negative breast cancer cells. Oncotarget, 4, 984-994. https://10.18632/oncotarget.1070.

1. SIRT1 activator for use in promoting and/or enhancing sperm capacitation process in mammals.

2. SIRT1 activator according to claim 1, wherein the SIRT1 is at a concentration from 5-30 μM.

3. SIRT1 activator according to claim 1, wherein the SIRT1 activator is at a concentration of 10 μM.

4. SIRT1 activator according to claim 1, wherein the SIRT1 activator is B-[2-Methoxy-5-[(1E)-3-oxo-3-(3,4,5-trimethoxyphenyl)-1-propen-1-yl]phenyl]boronic acid or YK-3-237.

5. SIRT1 activator according to claim 1 for use in assisted reproductive technology.

6. SIRT1 activator according to claim 5, wherein the assisted reproductive technology is carried out with low quality oocytes.

7. SIRT1 activator according to claim 5, wherein assisted reproductive technology is intra-uterine insemination, in vitro fertilization or intracytoplasmic sperm injection.

8. SIRT1 activator according to claim 1 for use in gradient sperm selection.

9. SIRT1 activator according to claim 1 for use in idiopathic infertility or in male infertility associated to sperm capacitation disorders.

10. SIRT1 activator according to claim 1, wherein it is combined with a further sperm promoter and/or enhancer.

11. SIRT1 activator according to claim 1, wherein it is combined with a commercially available sperm media, preferably sperm washing media, cryopreservation media, thawing media or combinations thereof.

12. SIRT1 activator according to claim 11, wherein the media comprises spermatozoa previously washed and diluted.

13. SIRT1 activator according to claim 1, wherein it induces maximum tyrosine phosphorylation levels on human spermatozoa after incubation in a capacitating media containing HCO₃ and a sterol removal component, in particular Bovine Serum Albumin.

14. SIRT1 activator according to claim 1 for use in humans.

15. SIRT1 activator according to claim 1 for use in non-human mammals, particularly equines, more particularly in horses.

16. Pharmaceutical compound comprising the SIRT1 activator according to claim 1.

17. Kit comprising the SIRT1 activator according to claim 1.
